(19) United States
(12) Patent Application Publication
(10) Pub. No.: US 2009/0041662 A1
(43) Pub. Date: Feb. 12, 2009

(54) Method for Monitoring Patient or Subject Compliance with Medical Prescriptions, and Formulation for Use in the Method

(76) Inventors: Irie Yasuo, Tokushima (JP); Anil Modak, Haverhill, MA (US); Tadashi Mukai, Tokushima (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/884,876

(22) PCT Filed: Feb. 22, 2006
(86) PCT No.: PCT/JP2006/303809

§ 371 Date: Aug. 22, 2007

Related U.S. Application Data
(63) Continuation-in-part of application No. 11/062,554, filed on Feb. 23, 2005.

(51) Int. Cl. A61K 51/04; C07C 57/02; G01N 33/497

(52) U.S. Cl. 424/1.81; 554/223; 73/23.3

(57) ABSTRACT

The present invention provides a highly accurate method for monitoring patient or subject compliance with a medication prescription by collecting a sample of the exhaled air. The method of the present invention is easy and little burdens the patient or subject.

The method comprises the steps of:
(i) prescribing a patient or subject ingestion of at least one biologically active agent and at least one carbon isotope-labeled lipid,
(ii) obtaining a sample of the patient's or subject's exhaled air,
(iii) measuring the ratio of the carbon isotope-labeled CO₂ relative to ¹²CO₂ in the sample; and
(iv) confirming the ingestion of the prescribed biologically active agent based on the ratio of the carbon isotope-labeled CO₂ relative to ¹²CO₂.

### TECHNICAL FIELD

The present invention relates to a method for monitoring patient or subject compliance with medical prescriptions. The present invention also relates to a formulation designed to enable monitoring of patient or subject compliance with medical prescriptions.

### BACKGROUND OF THE INVENTION ART

Patients examined in medical facilities are prescribed drugs by the doctor and given the medication at a pharmacy or clinic. Medical prescriptions need to be taken by the prescribed use and dose, and if they are used incorrectly, they may not only fail to exhibit the expected efficacy, but also cause harmful side effects to the patient. In order to make patients correctly understand the precautions regarding the use, side effects of medical prescriptions, medication instructions are given to the patients both orally and in writing.

In clinical trials performed in the developmental stage of drugs, the subjects are also given an explanation of side effects and other precautions to assure their safety, and instructed, both orally and in writing, to follow the prescribed use and dose in order to accurately assess the efficacy of the drugs.

Medication schedules for inpatients are complied relatively faithfully, since a nurse can manage the schedules by giving medication to the patient or by instructing the patient to use them at every prescribed time for administration, and the doctor can see the patient as required to examine the efficacy and side effects.

However, outpatients and clinical trial subjects are required to comply with their medication schedules on their own, and in a number of cases they do not take the medication correctly.

For example, outpatients sometimes forget to take medication, or stop medications on their own initiative due to side effects. In particular, when tuberculosis patients or AIDS patients who need to take a large amount of drugs daily fail to comply with their medication schedules or stop medications, they may not get sufficient efficacy.

In clinical trials of pharmaceutical drugs administered continuously, the subjects take the formulations home and take them according to a prescribed medication schedule. Thus, for the same reasons as for outpatients, the subjects may take the formulations incorrectly. Further, in such clinical trials, the subjects may incorrectly report the fact of failing to take medications, leading to inaccurate assessment and judgment of the efficacy of the drugs.

In order to improve such medical situations, systems have been developed which transmit notices to remind patients or subjects to take medicines via the Internet (Japanese Unexamined Patent Publication No. 1998-91700) or by cellular phones (Japanese Unexamined Patent Publication No. 2003-296454). Also developed are a medicine managing case that has a medicine management board (Japanese Unexamined Patent Publication No. 1997-237294), and a medication management-supporting device that is capable of sounding an alarm if the medicine box is opened at a time other than to take medicines, and capable of notifying the time for taking medicines (Japanese Unexamined Patent Publication No. 2003-310715). However, in any such measures, the patients or test subjects still need to take medicines on their own, and the drug compliance relies on the word of the patients or subjects.

Although it is possible to confirm compliance by measuring the drug levels in a blood or urine sample, collecting blood or urine samples burdens the patients or subjects them to pain and/or unpleasant procedures, and the measurements require a long time. Thus, such methods are neither easy nor accurate.

### SUMMARY OF THE INVENTION

An object of the present invention is to provide a method for easily and accurately monitoring patient or subject compliance with medical prescriptions. Specifically, the object of the present invention is to provide a method for monitoring patient or subject compliance with medical prescriptions easily, accurately, and with little burden on the patient or subject by collecting the exhaled air of the patient or subject. Another object of the present invention is to provide a formulation-designed to enable monitoring of patient or subject compliance with medical prescriptions.

The present inventors carried out extensive research to solve the above problems, and found that when an carbon isotope-labeled lipid and a biologically active agent are prescribed so as to be taken together, and ingested by a patient or subject, the carbon isotope-labeled lipid is metabolized in the body by β-oxidation, and carbon isotope-labeled carbon dioxide can be detected in the patient's or subject's exhaled air. The inventors further found that the carbon isotope-labeled carbon dioxide can be used as an index for monitoring the patient' or subject's compliance with medical prescriptions. The present invention was accomplished based on improvements on these findings.

The present invention provides the following methods for monitoring compliance with medical prescriptions.

1. A method of monitoring patient or subject compliance with a medical prescription comprising the steps of:
(i) prescribing a patient or subject ingestion of at least one biologically active agent and at least one carbon isotope-labeled lipid,
(ii) obtaining a sample of the patient's or subject's exhaled air,
(iii) measuring-the ratio of the carbon isotope-labeled CO₂ relative to ¹²CO₂ in the sample, and
(iv) confirming the ingestion of said prescribed biologically active agent based on the ratio of the carbon isotope-labeled CO₂ relative to ¹²CO₂.

2. A method according to Item 1, wherein the carbon isotope-labeled lipid is derived from one or more kinds of algae.

3. A method according to Item 1, wherein the carbon isotope-labeled lipid is a ¹³C-labeled lipid.

4. A method according to Item 1, wherein, in step (i), a pharmaceutical formulation comprising at least one biologically active agent and at least one carbon isotope-labeled lipid is prescribed.

The present invention encompasses methods of monitoring compliance with a medical prescription comprising steps (ii) to (iv) or steps (iii) and (iv) of the above methods. Thus, the present invention also provides the following methods.

5. A method of monitoring compliance with a medical prescription by a patient or subject prescribed at least one biologically active agent and at least one carbon isotope-labeled lipid, the method comprising the steps of:
(a) obtaining a sample of the patient's or subject's exhaled air,
(b) measuring the ratio of the carbon isotope-labeled CO₂-relative to ¹²CO₂ in the sample, and
(c) confirming the ingestion of said prescribed biologically active agent based on the ratio of the carbon isotope-labeled CO₂ relative to ¹²CO₂.

6. A method of monitoring compliance with a medical prescription by a patient or subject prescribed at least one biologically active agent and at least one carbon isotope-labeled lipid, the method comprising the steps of:
(1) measuring the ratio of the carbon isotope-labeled CO₂ relative to ¹²CO₂ in a sample obtained from the patient's or subject's exhaled air, and
(2) confirming the ingestion of said prescribed biologically active agent based on the ratio of the carbon isotope-labeled CO₂ relative to ¹²CO₂.

7. A method according to Item 5 or 6, wherein the carbon isotope-labeled lipid is derived from one or more kinds of algae.

8. A method according to Item 5 or 6, wherein the carbon isotope-labeled lipid is a ¹³C-labeled lipid.

9. A method according to Item 5 or 6, wherein, in step (i), a pharmaceutical formulation comprising at least one biologically active agent and at least one carbon isotope-labeled lipid is prescribed.

The present invention also provides the following formulations that enable monitoring of patient or subject compliance with medical prescriptions.

10. An oral formulation for monitoring patient or subject compliance with a medical prescription, the formulation comprising at least one carbon isotope-labeled lipid.

11. An oral formulation according to Item 6, wherein the carbon isotope-labeled lipid is derived from one or more kinds of algae.

12. An oral pharmaceutical formulation comprising at least one carbon isotope-labeled lipid and at least one biologically active agent.

13. An oral pharmaceutical formulation according to Item 12, wherein the carbon isotope-labeled lipid is derived from one or more kinds of algae.

The present invention further provides the following use of a carbon isotope-labeled lipid.

14. Use of a carbon isotope-labeled lipid for production of an oral formulation for monitoring patient or subject compliance with a medical prescription.

15. Use of a carbon isotope-labeled lipid for production of an oral pharmaceutical formulation that enables monitoring of patient or subject compliance with a medication prescription.

16. Use of a carbon isotope-labeled lipid for monitoring subject or patient compliance with a medication prescription of ingestion of an oral pharmaceutical formulation.

### BRIEF DESCRIPTION OF THE DRAWINGS

FIG. 1 shows the results of Test Example 1 in which the formulation of Example 1 was ingested at 8 hour intervals for 3 days, and the ¹³CO₂/¹²CO₂ concentration ratio (δ¹³C value) in the exhaled air was measured over time.

FIG. 2 shows the results of Test Example 2 in which the formulation of Example 3 was ingested at 24 hour intervals for 3 days, and the ¹³CO₂/¹²CO₂ concentration ratio (δ¹³C value) in the exhaled air was measured over time.

FIG. 3 shows the results of the ingestion of the formulation of Examples 3 by two healthy volunteers (Vlt-1 and Vlt-2) at 24 hour intervals (everyday before breakfast) for 3 days, and the ¹³CO₂/¹²CO₂ concentration ratio (δ¹³C value) in their exhaled air was measured over time. In the figure, ◯ shows the result of ingestion of the formulation of Example 3 by Vlt-1; and , the result of ingestion of the formulation of Example 3 by Vlt-2.

### DISCLOSURE OF THE INVENTION

The present invention is described below in detail.

In the method of the present invention, the targets of monitoring are patients or subjects. As used herein, “patient” is intended to mean someone who needs medication for treating a disease and/or alleviating symptoms; and “subject” is intended to mean, as well as someone who needs medication for preventing and/or diagnosing a disease, someone who takes medicine for confirming the efficacy of the medicine in clinical trials and/or other tests.

The steps of the method of the present invention are described below.

### Step (i)

In the method of the present invention, a patient or subject is prescribed ingestion of at least one carbon isotope-labeled lipid together with at least one biologically active agent (Step (i)).

A biologically active agent is an agent useful for preventing, treating and/or diagnosing humans and/or non-human animals. Biologically active agents encompass precursors that can be readily converted to biologically active agents by enzymolysis or hydrolysis, such as prodrugs. Examples of biologically active agents include anti-infectives (e.g., antibiotics, antifungals and antiviral drugs), antitumor drugs, immunomodulators (e.g., antihistamines, immunopotentiators and immunosuppressants), antianginal drugs, analgesics, antipyretics, hypnotics, sedatives, antacids, anti-inflammatory drugs, antimaniac agents, vasodilators, psychotropic drugs, anesthetics, stimulants, antidiarrheals, antiemetics, growth promoters, antispasmodics, neuromuscular drugs, vasopressors, hypotensors, diuretics, cytotoxic compounds, anticonvulsants, antarthritics, utero relaxants, anti-obesity drugs, anthelmintics, laxatives, hormones, vaccines, vitamins, dietary supplements, etc. Such biologically active agents can be used singly or in combination. The biologically active agent(s) to be used is suitably selected depending on the symptoms and diseases of the patient, and other factors.

Carbon isotope-labeled lipids are lipids in which at least one carbon atom is substituted by a carbon isotope. The carbon isotope-labeled lipid used in the present invention is decomposed by β-oxidation in vivo, to cause the decomposed product to appear in the exhaled air as isotopically labeled carbon dioxide, which is used as an index for monitoring patient or subject compliance with a medical prescription. Therefore, the carbon isotope-labeled lipid preferably contains a carbon isotope in a proportion sufficient to allow the metabolized product, isotopically labeled carbon dioxide, to be readily detected. Specifically, the proportion of carbon isotope in the total carbon of the carbon isotope-labeled lipid is, for example, at least 10%, preferably at least 50%, and more preferably at least 90%.

The carbon isotope may be, for example, ¹³C, ¹¹C or ¹⁴C, of which ¹³C is nonradioactive and thus preferable from the safety viewpoint.

The carbon isotope-labeled lipid is not limited in structure, as long as it is metabolized in the body and discharged from the body as carbon dioxide. Examples of such lipids include glycerides, phospholipids, glycolipids, fatty acids, etc. Specifically, glycerides include monoglycerides, diglycerides, triglycerides, polyglycerides, etc. Phospholipids include phosphatidylethanolamine, phosphatidic acid, phosphatidylcholine, phosphatidylserine and other glycerophospholipids; sphingomyelin and other sphingophospholipids; etc. Glycolipids include cerebroside and other sphingoglycolipids; glyceroglycolipid; etc. Fatty acids include short chain fatty acids (carbon number: 2 to 4), medium chain fatty acids (carbon number: 5 to 10), long chain fatty acids (carbon number: 11 or more), etc.

Hydrocarbon chains of the carbon isotope-labeled lipids may be saturated or unsaturated. The number of carbon atoms in a hydrocarbon chain is not limited, and may be, for example, 1 to 36, preferably 10 to 30, and more preferably 16 to 18. Since the end of the hydrocarbon chain of the lipid is sequentially severed by β-oxidation in the body, when ingested, a lipid that has a hydrocarbon chain with the above carbon number causes carbon dioxide with lipid-derived carbon to be continuously discharged in the exhaled air.

Of such lipids, preferably usable is at least one fatty acid selected from the group consisting of lauric acid, palmitic acid, stearic acid, palmitoleic acid, oleic acid and linolic acid; and/or at least one member selected from the group consisting of glycerides, phospholipids and glycolipids, each containing at least one acyl group derived from said at least one fatty acid. Particularly preferable is at least one fatty acid selected from the group consisting of palmitic acid, palmitoleic acid, oleic acid and linolic acid; and/or at least one member selected from the group consisting of glycerides, phospholipids and glycolipids, each containing at least one acyl group derived from said at least one fatty acid. More particularly preferable is at least one triglyceride containing acyl groups derived from at least one fatty acid selected from the group consisting of palmitic acid, palmitoleic acid, oleic acid and linolic acid.

The above-mentioned lipids may be used singly or in combination.

The carbon isotope-labeled lipid can be prepared by a hitherto known process.

A specific example of a process for preparing a ¹³C-labeled lipid comprises collecting a lipid from one or more kinds of algae cultured in a ¹³C-labeled carbon dioxide environment, which can be created by aerating the culture medium with ¹³C-labeled carbon dioxide. The proportion of ¹³C-labeled carbon dioxide in the total carbon dioxide used for the aeration correlates to the proportion of ¹³C-labeled carbon in the total carbon of the ¹³C-labeled lipid to be collected from the cultured algae, and thus is suitably determined to obtain a desired ¹³C-labeled carbon proportion. The cultured algae are subjected to a standard separation and purification process to collect the lipid. Algae usable for preparing the ¹³C-labeled lipid are not limited, and include blue-green algae and other species. Such alga-derived ¹³C-labeled lipids can be produced at low cost and are highly safe, and therefore are preferably used in the method of the present invention.

The biologically active agent and carbon isotope-labeled lipid, when prescribed for a patient or subject, may be formulated separately or together. That is, in the former case, two formulations, comprising the biologically active agent and carbon isotope-labeled lipid, respectively, are prescribed, and in the latter case, a single formulation comprising both the biologically active agent and the carbon isotope-labeled lipid is prescribed. To avoid failure of ingesting one of the biologically active agent and the carbon isotope-labeled lipid, it is preferable to prepare a formulation comprising both the biologically active agent and the carbon isotope-labeled lipid.

The dose of the biologically active agent is suitably selected according to the type of the agent, patient's symptoms, patient's or subject's sex and age, metabolic capacity etc., and is generally, for example, about 10 to 3000 mg per day for an adult human, and either a fixed dose or variable dose (mg/kg or mg/BSA) for an adult human.

The dose of the carbon isotope-labeled lipid is suitably selected according to the type of the carbon isotope-labeled lipid, patient's or subject's sex and age, timing of collecting exhaled air sample(s), number of times of ingestion per day, etc., and is, for example, usually about 1 to 1000 mg, preferably 10 to 500 mg, and more preferably 100 to 200 mg per day, for an adult human.

Described below in detail are the “pharmaceutical formulation comprising a biologically active agent”, “formulation comprising a carbon isotope-labeled lipid”, and “pharmaceutical formulation comprising a biologically active agent and a carbon isotope-labeled lipid” for use in Step (i).

### Pharmaceutical Formulation Comprising Biologically Active Agent

The pharmaceutical formulation comprising a biologically active agent is not limited in form, as long as it can be taken orally, and may be in a powder, granule, tablet, pill or other solid form, or in a liquid form. It may also be a capsule or a coated formulation. The formulation can be formulated by a standard method: specifically, by formulating an effective amount of biologically active agent and, as required, suitable amounts of pharmaceutically acceptable additives and/or carriers into a formulation.

The proportion of the biologically active agent in the pharmaceutical formulation can be suitably selected according to the dose of the biologically active agent.

### Formulation Comprising Carbon Isotope-Labeled Lipid

The formulation comprising at least one carbon isotope-labeled lipid is also not limited in form, as long as it can be taken orally. It may be in a powder, granule, tablet, pill or other solid form, or in a liquid form. It may also be a capsule or a coated formulation. The formulation can be formulated by a standard method: specifically, by formulating an effective amount of carbon isotope-labeled lipid and, as required, suitable amounts of pharmaceutically acceptable additives and/or carriers into a formulation. More specifically, a solid formulation can be obtained by, for example, causing the carbon isotope-labeled lipid to be adsorbed on an excipient or like additive, which is then subjected to a treatment such as grinding. Alternatively, a liquid formulation can be obtained by, for example, formulating the carbon isotope-labeled lipid together with a surfactant by a standard process.

In this formulation, it is preferable to suitably control the release rate of the carbon isotope-labeled lipid according to the interval of ingestion, timing of collecting exhaled air sample(s), metabolism rate of the carbon isotope-labeled lipid, etc. The release rate can be controlled by a standard method, specifically, by adding to the formulation a water-insoluble substance, highly viscous water-soluble substance and/or like release rate controlling carrier, and/or by coating the formulation, so as to control the release rate of the isotopically labeled lipid depending on the amount of the release rate-controlling carrier and/or coating layer.

Specific examples of release controlling carriers include ethylcellulose, aminoalkylmethacrylate copolymer RS, hydrogenated oils, carnauba wax, glyceryl monostearate and other water-insoluble substances; hydroxypropylcellulose, hydroxypropylmethylcellulose, methylcellulose, xanthan gum, locust bean gum, alginic acids and other highly viscous water-soluble substances; etc.

The proportion of the carbon isotope-labeled lipid in the formulation can be suitably selected according to the dose of the carbon isotope-labeled lipid.

The formulation is useful as an oral formulation for monitoring patient or subject compliance with medical prescriptions.

### Pharmaceutical Formulation Comprising Biologically Active Agent and Carbon Isotope-Labeled Lipid

The pharmaceutical formulation comprising at least one biologically active agent and at least one carbon isotope-labeled lipid is also not limited in form, as long as it can be orally taken. It may be in a powder, granule, tablet, pill or other solid form, or in a liquid form. It may also be a capsule or a coated formulation. The formulation is produced by a standard method, specifically, by formulating effective amounts of the biologically active agent and carbon isotope-labeled lipid and, as required, suitable amounts of pharmaceutically acceptable additives and/or carriers into a formulation. More specifically, a solid formulation can be obtained by, for example, causing the carbon isotope-labeled lipid to be adsorbed on an excipient or like additive, which is then subjected to a treatment such as grinding and mixed with the biologically active agent, and forming the mixture into a formulation by a standard process. Alternatively, a liquid formulation can be obtained by, for example, produced, by a standard process, the carbon isotope-labeled lipid and biologically active agent together with a surfactant and/or other additives.

It is preferable to suitably control the release rate of the carbon isotope-labeled lipid according to the interval of ingestion, timing of collecting exhaled air sample(s), metabolism rate of the carbon isotope-labeled lipid, etc. The release rate can be controlled in the same manner as for the formulation comprising the carbon isotope-labeled lipid, specifically, by adding a release rate-controlling carrier to the formulation and/or by coating the formulation, so as to control the release rate of the formulation as a whole. Alternatively, the carbon isotope-labeled lipid, before being mixed with the biologically active agent, may be blended with a release rate controlling carrier and/or provided with coating, so as to control the release rate of only the carbon isotope-labeled lipid.

The proportions of the biologically active agent and carbon isotope-labeled lipid in the formulation can be suitably determined according to the doses thereof.

The formulation is useful as a pharmaceutical formulation that exhibits pharmacological activity due to the biologically active agent and enables monitoring of patient or subject compliance with a medication prescription due to the carbon isotope-labeled lipid.

Examples of additives and carriers that can be added to the above formulations include excipients, binders, pH modifiers, disintegrators, absorption promoters, lubricants, coloring agents, corrigents, flavors, perfumes, aqueous media, etc. Specific examples include lactose, saccharose, mannitol, sodium chloride, glucose, calcium carbonate, kaolin, crystalline cellulose, silicates and other excipients; water, ethanol, simple syrup, glucose solutions, starch solutions, gelatin solutions, carboxymethylcellulose, sodium carboxymethylcellulose, shellac, methylcellulose, hydroxypropylmethylcellulose, hydroxypropylcellulose, polyvinylpyrrolidone, polyvinyl alcohol, gelatin, dextrin, pullulan, and other binders; citric acid, citric anhydride, sodium citrate, sodium citrate dehydrate, anhydrous sodium monohydrogenphosphate, anhydrous sodium dihydrogenphosphate, sodium hydrogenphosphate, and other pH modifiers; carmellose calcium, low-substituted hydroxypropylcellulose, carmellose, croscarmellose sodium, carboxymethyl starch sodium, crospovidone, polysorbate-80, and other disintegraters; quaternary ammonium bases, sodium lauryl sulfate, and other absorption promoters; purified talc, stearic acid salts, polyethylene glycol, colloidal silicic acid, sucrose esters of fatty acids, and other lubricants; yellow iron oxide, yellow iron sesquioxide, iron sesquioxide, β-carotene, titanium oxide, food colors (e.g., Food Blue No. 1), copper chlorophyll, riboflavin, and other coloring agents; ascorbic acid, aspartame, Hydrangeae Dulcis Folium, sodium chloride, fructose, saccharin, powdered sugar, and other corrigents; water, physiological saline, and other aqueous media; etc.

### Step (ii)

Subsequently, in the method of the present invention, after the prescribed time for ingestion of the formulation(s), a sample of the patient's or subject's exhaled air is collected (Step (ii)).

The timing of sample collection is not limited as long as it is after the prescribed time for ingestion of the formulation(s), and is suitably determined according to the type of carbon isotope-labeled lipid, metabolism rate of the carbon isotope-labeled lipid in the body, etc. Specifically, the timing may be 0 to 24 hours after the prescribed time for ingestion of the formulation(s).

Step (iii)

Subsequently, the concentration ratio of the carbon isotope-labeled CO₂ to ¹²CO₂ (hereafter referred to as “δ carbon isotope-labeled CO₂ value”) in the collected sample of the exhaled air is measured (Step (iii)).

The method for measuring the labeled CO₂ contained in the exhaled air sample is selected depending on whether the isotope used is radioactive or nonradioactive. Conventional analytical techniques, such as liquid scintillation counting, mass spectrometry, infrared spectrometry, emission spectrometry, and magnetic resonance spectrum analysis can be used for the measurement. Preferable are infrared spectrometry and mass spectrometry from the viewpoint of measurement accuracy. When ¹³C is used as the carbon isotope, an infrared spectrometer (POCone or UBiT-IR300, Otsuka Electronics Co., Ltd.) can be used for simple and easy measurement.

### Step (iv)

The measured δ carbon isotope-labeled CO₂ value is used to confirm whether the patient or subject has ingested the biologically active agent (Step (iv)). If the carbon isotope-labeled lipid has been ingested, it is decomposed in the body to cause carbon isotope-labeled CO₂ to be discharged in the exhaled air. As a result, δ carbon isotope-labeled CO₂ value in the exhaled air sequentially increases. If the carbon isotope-labeled lipid has not been ingested, as per the dosing requirements less carbon isotope-labeled CO₂ is discharged in the exhaled air, so that δ carbon isotope-labeled CO₂ value is lowered.

It is preferable, before administration of the biologically active agent, to have the patient or subject to ingest a formulation comprising only the carbon isotope-labeled lipid according to the same medication schedule as for the biologically active agent, in order to measure the δ carbon isotope-labeled CO₂ value in the exhaled air over time and prepare a standard curve for the δ carbon isotope-labeled CO₂ value in the exhaled air according to the schedule. By comparing the standard curve with the curve of the δ carbon isotope-labeled CO₂ value obtained after the prescribed time for ingestion of the biologically active agent and carbon isotope-labeled lipid, it is possible to confirm not only whether the biologically active agent has been ingested or not, but also whether it has been ingested at a suitable time.

In this manner, the method of the present invention is capable of confirming the situation of medicine ingestion, making it possible to monitor compliance with medication prescriptions.

### INDUSTRIAL APPLICABILITY

The end of a hydrocarbon chain of the carbon isotope-labeled lipid used in the present invention is sequentially metabolized by β-oxidation in the body, and converted into carbon dioxide. Thus, after the carbon isotope-labeled lipid has been ingested, carbon isotope-labeled CO₂ is continuously discharged in the exhaled air.

The method of the present invention uses carbon isotope-labeled CO₂ as an index of ingestion of a drug, and enables very easy confirmation of whether the patient or subject has ingested the biologically active agent correctly. If a carbon isotope-labeled sugar or amino acid is used in place of the carbon isotope-labeled lipid, the metabolism rate is too rapid to cause continuous discharge of carbon isotope-labeled CO₂ in the exhaled air, making it difficult to monitor patient or subject compliance with medical prescriptions.

### EXAMPLES

The following Examples and Test Examples are intended to illustrate the present invention, and in no way to limit the scope of the invention.

### Reference Example 1

### Production of Alga-Derived ¹³C-Labeled Lipid

A carbon isotope-labeled lipid was prepared by a known process. Specifically, algal cells (blue-green alga species) were cultivated in the presence of ¹³C-labeled carbon dioxide. Then, the cells were collected by centrifugation and subjected to extraction with a mixture of MeOH and CH₂Cl₂ to obtain a crude product of an carbon isotope-labeled lipid. The crude product (1.45 kg) was dissolved in 9 L of aqueous solution of 1.08 kg of sodium hydroxide. The solution was heated at 50° C. for several days, then heated under reflux for 8 hours, allowed to cool to room temperature, and subjected to extraction with diethyl ether (2 L) five times to remove impurities. Concentrated hydrochloric acid was added to the aqueous layer to adjust the pH to 2, extraction with diethyl ether (2 L) was carried out 5 times to collect the desired product in the organic layer. The organic layer was evaporated to dryness, and dissolved in diethyl ether (5 L). Activated carbon (30 g) was added to the solution, and the mixture was stirred at room temperature for 2 hours, and subjected to silica gel chromatography (SiO₂: 3 to 4 L) for decolorization. The collected ether solution was evaporated to dryness, to thereby give 450 to 500 g of the final product.

### Example 1

A 100 mg portion of the ¹³C-labeled lipid obtained in Reference Example 1 was packed in gelatin capsules (the Japanese pharmacopoeia, size #0, Matsuya Co., Ltd.) to obtain a capsule formulation.

### Example 2

A 100 mg portion of the ¹³C-labeled lipid obtained in Reference Example 1 was placed in a glass bottle and dissolved by ultrasonic treatment in ethanol (1.0 ml). The ethanol solution was added to 50 mg of calcium silicate (FLORITE-RE, Eisai Co., Ltd.) placed in a mortar, and mixed using a pestle to allow the lipid to adsorb on calcium silicate. After air-drying for 20 minutes, 200 mg of granulated lactose (DILACTOSE, Freund) was added and mixed. The mixture was further mixed with 50 mg of low substituted hydroxypropylcellulose (LH-31, Shin-Etsu Chemical Co., Ltd.) to prepare a sample for tablet pressing. A 400 mg portion of the sample was pressed (1 mm/sec) using AUTOGRAPH AG-1 (Shimazu Co., Ltd.) equipped with flat face punches of 9.5 mm diameter, at a compressional force of 1 ton to form immediate-release tablets.

### Example 3

Sustained-release tablets were prepared in the same manner as in Example 2, with the exception that low-substituted hydroxypropylcellulose was replaced with highly viscous hydroxypropylmethylcellulose (Metorose 90SH4000, Shin-Etsu Chemical Co., Ltd.).

### Example 4

Sustained-release tablets with a faster isotope carbon-labeled lipid release rate than those of Example 3 were prepared in the same manner as in Example 2, with the exception that the amount of the highly viscous hydroxypropylmethylcellulose was reduced to 25 mg.

### Test Example 1

The following procedure was performed to measure the change in δ¹³CO₂ value in the exhaled air of one healthy volunteer by ingestion of the capsule formulation of Example 1.

A baseline breath sample was first collected in an aluminium-lined bag with a capacity of about 1.2 L. After a 10 hour fasting, the capsule formulation of Example 1 was orally administered at 8 hour intervals (6:00 a.m., 2:00 p.m. and 10:00 p.m.) everyday for 3 days. Samples of the exhaled air were collected 24, 48 and 72 hours after the first administration in aluminium-lined bags with a capacity of about 300 ml (Schedule 1). After cessation of medication for 1 week, the capsule formulation of Example 1 was orally administered again at 8 hour intervals for 3 days, during which, however, the formulation was not administered 16, 40, 64 and 72 hours after the first administration (Schedule 2).

The concentrations of ¹³CO₂ and ¹²CO₂ in the exhaled air samples were measured using an infrared spectrometer (UbiT-IR300, Otsuka Electronics Co., Ltd.). A δ¹³CO₂ value (DOB), which represents the change in the ¹³CO₂/¹²CO₂ ratio in the exhaled air samples collected before and after the ingestion of the formulation, was calculated according to the following equation.

DOB=(¹³CO₂/¹²CO2)_{post dose}−(¹³CO₂/¹²CO)_{baseline sample}

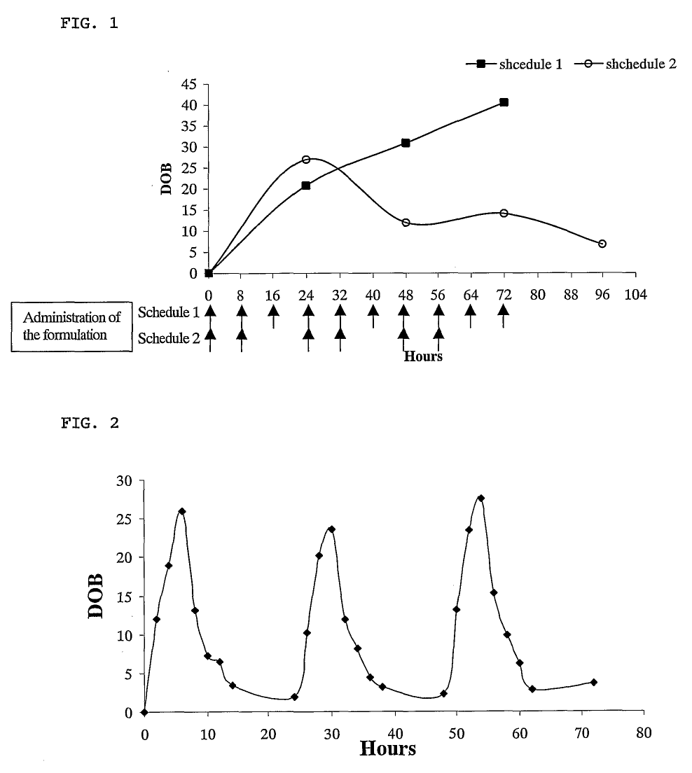

FIG. 1 shows the results. When the capsule formulation of Example 1 was administered 3 times per day at 8 hour intervals (Schedule 1), the δ¹³CO₂ value measured every 24 hours varied substantially linearly. In contrast, when the formulation was not administered 16, 40, 64 and 72 hours after the first administration (Schedule 2), the δ¹³CO₂ value greatly decreased. The results demonstrate that the situation of medicine ingestion can be confirmed by administering the formulation of Example 1 and measuring the δ¹³CO₂ value in the exhaled air.

### Test Example 2

The following procedure was performed to measure the change in δ¹³CO₂ value in the exhaled air of one healthy volunteer by ingestion of the formulation of Example 3.

A baseline breath sample was first collected in an aluminium-lined bag with a capacity of about 3 L. After a 10 hour fasting, the formulation of Example 3 was orally administered at 24 hour intervals (at 6:00 a.m. everyday) for 3 days. Samples of the exhaled air were collected immediately after the administration, every 2 hours from 8:00 a.m. to 10:00 p.m., and 72 hours after the first administration, in aluminium-lined bags with a capacity of about 300 ml. The δ¹³CO₂ values of the exhaled air samples were measured in the same manner as in Test Example 1.

FIG. 2 shows the results. The results show that when the formulation of Example 3 was administered for 3 days, the δ¹³CO₂ value varied similarly every day during the 3 days, demonstrating that the compliance of medicine ingestion can be confirmed by measuring the δ¹³CO₂ value.

### Test Example 3

The following procedure was performed to measure the change in δ¹³CO₂ value in the exhaled air of two healthy volunteers (Vlt-1 and Vlt-2) by ingestion of the formulations of Examples 3 and 4.

Baseline breath samples were first collected in aluminium-lined bags with a capacity of about 1.2 L. After a 10 hour fasting, the formulation of Example 3 was orally administered at 24 hour intervals (at 6:00 a.m. everyday) for 3 days. Subsequently, after cessation of medication for at least 1 week, the formulation of Example 4 was orally administered at 24 hour intervals (at 6:00 a.m. everyday) for 3 days. During the administration period of each formulation, samples of the exhaled air were collected at 24 hour intervals in aluminium-lined bags with a capacity of about 300 ml. The δ¹³CO₂ values in the collected exhaled air samples were measured in the same manner as in Test Example 1.

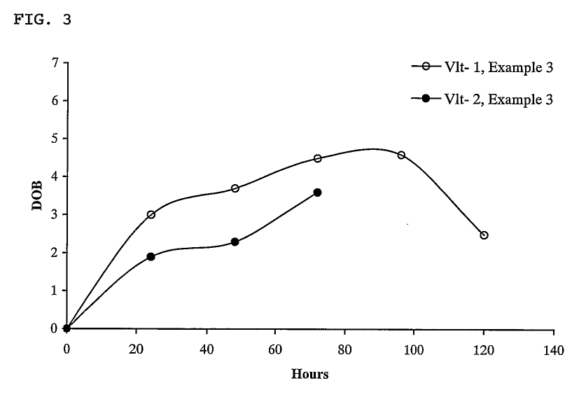

FIG. 3 shows the results of the ingestion of the formulation of Example 3 by two volunteers. FIG. 3 shows that, during the administration period of the formulation of Example 3, the δ¹³CO₂ value increased substantially linearly from 24 hours, through 48 hours, to 72 hours after the first administration, demonstrating that this formulation is especially suitable for monitoring the situation of ingestion of medicines that are used continuously.

Similarly, during the administration period of the formulation of Example 4, the δ¹³CO₂ value also gradually increased. This result demonstrates that the formulation of Example 4, which is sustained-release tablets, enables monitoring of the situation of ingestion of medicines that are administered at 24 hour intervals.

1. A method of monitoring patient or subject compliance with a medical prescription comprising the steps of:
(i) prescribing a patient or subject ingestion of at least one biologically active agent and at least one carbon isotope-labeled lipid,
(ii) obtaining a sample of the patient's or subject's exhaled air,
(iii) measuring the ratio of the carbon isotope-labeled CO₂ relative to ¹²CO₂ in the sample, and
(iv) confirming the ingestion of said prescribed biologically active agent based on the ratio of the carbon isotope-labeled CO₂ relative to ¹²CO₂.

2. A method according to claim 1, wherein the carbon isotope-labeled lipid is derived from one or more kinds of algae.

3. A method according to claim 1, wherein the carbon isotope-labeled lipid is a ¹³C-labeled lipid.

4. A method according to claim 1, wherein, in step (i), a pharmaceutical formulation comprising at least one biologically active agent and at least one carbon isotope-labeled lipid is prescribed.

5. A method of monitoring compliance with a medical prescription by a patient or subject prescribed at least one biologically active agent and at least one carbon isotope-labeled lipid, comprising the steps of:
(1) measuring the ratio of the carbon isotope-labeled CO₂ relative to ¹²CO₂ in a sample obtained from the patient's or subject's exhaled air, and
(2) confirming the ingestion of said prescribed biologically active agent based on the ratio of the carbon isotope-labeled CO₂ relative to ¹²CO₂.

6. A method according to claim 5, wherein the carbon isotope-labeled lipid is derived from one or more kinds of algae.

7. A method according to claim 5, wherein the carbon isotope-labeled lipid is a ¹³C-labeled lipid.

8. A method according to claim 5, wherein, in step (1), a pharmaceutical formulation comprising at least one biologically active agent and at least one carbon isotope-labeled lipid is prescribed.

9. An oral formulation for monitoring patient or subject compliance with a medication prescription, the formulation comprising at least one carbon isotope-labeled lipid.

10. An oral formulation according to claim 9, wherein the carbon isotope-labeled lipid is derived from one or more kinds of algae.

11. An oral pharmaceutical formulation comprising at least one carbon isotope-labeled lipid and at least one biologically active agent.

12. An oral pharmaceutical formulation according to claim 11, wherein the carbon isotope-labeled lipid is derived from one or more kinds of algae.

13. Use of a carbon isotope-labeled lipid for production of an oral formulation for monitoring patient or subject compliance with a medical prescription.

14. Use of a carbon isotope-labeled lipid for production of an oral pharmaceutical formulation that enables monitoring of patient or subject compliance with a medication prescription.

15. Use of a carbon isotope-labeled lipid for monitoring subject or patient compliance with a medication prescription of ingestion of an oral pharmaceutical formulation.
